(19) United States
(12) Patent Application Publication
(10) Pub. No.: US 2023/0404969 A1
(43) Pub. Date: Dec. 21, 2023

(54) COMPOSITIONS AND METHOD FOR EFFECTIVE MANAGEMENT OF PERITONITIS

(71) Applicants: Muhammed Majeed, Bangalore (IN); Kalyanam Nagabhushanam, East Windsor, NJ (US); Sarang Bani, Bangalore (IN); Anjali Pandey, Bangalore (IN)

(72) Inventors: Muhammed Majeed, Bangalore (IN); Kalyanam Nagabhushanam, East Windsor, NJ (US); Sarang Bani, Bangalore (IN); Anjali Pandey, Bangalore (IN)

(21) Appl. No.: 17/944,762

(22) Filed: Sep. 14, 2022

(30) Foreign Application Priority Data
Jun. 20, 2022 (IN) 202241035136

(51) Int. Cl. A61K 31/366; G16H 20/10; G16H 50/30; A61K 9/16; A61K 9/48; A61P 29/00

(52) U.S. Cl. CPC A61K 31/366; G16H 20/10; G16H 50/30; A61K 9/16; A61K 9/48; A61P 29/00

(57) ABSTRACT

The disclosed invention relates to a method comprising composition for effective management of peritonitis in a subject. The invention also discloses a method comprising composition for preventing peritonitis mediated multi organ dysfunction and management of inflammation in a subject. The composition disclosed essentially consists of Witharione.

### CROSS REFERENCE TO RELATED APPLICATIONS

This is a non-provisional application claiming priority from Indian application no. 202241035136, filed on 20 Jun. 2022, the contents of which are incorporated herein by reference.

### FIELD OF INVENTION

The invention in general relates to a method and compositions for use in therapeutic management of peritonitis in a subject. The invention also discloses a composition and method for preventing peritonitis mediated multi organ dysfunction. Still more specifically, the invention discloses the composition consisting essentially of Withanone.

### BACKGROUND OF INVENTION

Inflammation is a response to factors such as pathogens, damaged cells, or toxic compounds and the host's response to this acts as a defense to maintain homeostasis. Peritonitis is an inflammation of the peritoneum, the tissue that lines the inner wall of the abdomen and covers and supports most of abdominal organs. It is commonly caused by gastrointestinal rupture, perforation. Typical causes include spontaneous bacterial or fungal infection, visceral perforation or damage, inflammation, appendicitis, inflammatory bowel diseases, and feline infectious peritonitis virus. Studies have shown that intraperitoneal inoculation of B. fragilis in the gut secretes lipopolysaccharide (LPS), which in turn promotes inflammation leading to the production of IL-1β, IL-6, IL-6, TNF-α via activation of TLR-2 (Lobo et al. The interplay between microbiola and inflammation: lessons from peritonitis and sepsis, Clin. Trans. immunology, 2016; 5; e90). The three ways by which the peritoneal cavity responds to an infection, 1) absorption of bacteria by diaphragmatic stomata, 2) destruction of bacteria by complement cascade and phagocytes, 3) Localization of infection as abscess. The first line of defense by the peritoneum is to clear the infection by lymphatics, accompanied by activation of complement cascade by invasion of polymorphonuclear neutrophils (PMNs) and macrophages. Initial symptoms of peritonitis are typically poor appetite and nausea and a dull abdominal ache that quickly turns into persistent, severe abdominal pain. Other signs and symptoms related to peritonitis may include: Abdominal tenderness or distention, chills, fever, fluid in the abdomen, not passing any urine, or passing significantly less urine than usual, difficult bowel movement, and vomiting. There are different types of peritonitis, primary, secondary and tertiary. Primary resulting from bacterial translocation, hematogenous spread, or the iatrogenic contamination of the abdomen. Whereas the secondary is due to the contamination of the peritoneum by spillage from the GI tract or urogenital tract or other organs, essentially bacterial pathogens traverse into peritoneal cavity leading to inflammatory response, sepsis, multi organ failure, and death. Peritonitis, which persists after 48 hours is tertiary (Ross et al. Secondary peritonitis: principles of diagnosis and intervention, BMJ, 2018; 361; k1407; Mabewa et al. Etiology, treatment outcome and prognostic factors among patients with secondary peritonitis at Bugando Medical Centre, Mwanza, Tanzania; World Journal of Emergency Surgery (2015) 10:47). The treatment involves decreasing the cytokine release of IL-1β, TNF-α, and IL-6. Many hypothesis have emerged on the possible role of peroxisome proliferator-activated receptor (PPARγ) as anti-inflammatory through NF-κB pathway and ERK/CDK5 pathway (Zhang et al. PPAR-γ agonist rosiglitazone ameliorates peritoneal deterioration in peritoneal dialysis rats with LPS-induced peritonitis through up-regulation of AQP-1 and ZO-1. Biosci Rep. 2018; 38; BSR20180009; Lin et al. Magnolol ameliorates lipopolysaccharide-induced acute lung injury in rats through PPAR-γ-dependent inhibition of NF-kB activation. Int Immunopharmacol 2015;28:270-8; Li et al. Lipopolysaccharide-induced inflammation in human peritoneal mesothelial cells is controlled by ERK1/2-CDK5-PPARγ axis. Ann Transl Med 2021 :9(10):850.). The current therapy for peritonitis involves either surgery or treatment with antimicrobial agents. However, the search for anti-inflammatory agents for peritonitis with minimal side effects, traditionally associated with other anti-inflammatory agents, is the way forward.

Acharya et al. demonstrated use of Withania somnifera (WS) as a whole extract for treating endotoxin induced peritonitis. WS consists of Withanolide A, Withastramnolide, Withanone, 27-Hydroxy Withanone, Withaferin A, Withanoside IV and each of these are known for plethora of activities. The use of combination of different agents would have immunosuppressive effects or other unwanted side effects. Moreover, the combination of different molecules would also diffuse the potential of an individual molecule.
Anjali et al. have documented the use of Withanone, isolated from the root of WS, for neuroprotective effects by mitigating inflammation (Anjali et al. Multifunctional neuroprotective effect of Withanone, a compound from Withania somnifera roots in alleviating cognitive dysfunction; Cytokine 2017). However, it is well known in the state of art that inflammation is disease and organ specific wherein there is a difference in the source, effect and intensity of inflammatory pathways that trigger the onset of the disease. Thus, it is a well-established fact that peritonitis is different from neurological diseases, driven my multitude of factors such as inflammation, bacterial infection, etc. A person skilled in the art will also realize that a single moiety may not be able to treat all the factors diving the disease. Hence, there is still an unmet industrial need to find a safe, effective natural molecule to address all the causative factors of peritonitis. The present invention solves the above mentioned need by disclosing the potential of withanone for the treatment of peritonitis.

### OBJECTIVES OF THE INVENTION

It is the main objective of the invention to disclose a method and composition in the therapeutic management of peritonitis in a mammal, administering an effective dose of a composition consisting essentially of Withanone.

In yet another main objective of the invention to disclose a method and composition for therapeutically managing inflammation in a subject, treating the subject with an effective dose of a composition consisting essentially of Withanone.

In another main objective of the invention to disclose a method and composition for preventing peritonitis mediated multi organ dysfunction in a subject, treating the subject with an effective dose of a composition consisting essentially of Withanone

### SUMMARY

The invention broadly solves the aforementioned problems mentioned in the background by covering a method and composition for use in therapeutic management of peritonitis in a subject. The invention also discloses a composition and method for preventing peritonitis mediated multi organ dysfunction. Still more specifically, the invention discloses the composition consisting essentially of Withanone.

The first aspect of the invention relates to a composition consisting essentially of Withanone for use in the therapeutic management of peritonitis in a mammal.

In yet another aspect of the invention covers a composition consisting essentially of Withanone for therapeutically managing inflammation in a subject.

In another aspect of the invention covers a composition consisting essentially of Withanone for use in preventing peritonitis mediated multi organ dysfunction in a subject.

In another aspect of the invention covers a method of therapeutic management of peritonitis in a mammal, said method comprising steps of a) identifying a mammal with peritonitis; and b) administering an effective dose of a composition consisting essentially of Withanone to said mammal, to alleviate the symptoms of peritonitis.

In yet another aspect of the invention covers a method for therapeutically managing inflammation in a subject, comprising steps of a) Identifying the subject with inflammation; and b) treating the subject with an effective dose of a composition consisting essentially of Withanone, to alleviate the expression of markers associated with inflammation and inhibiting the formation of membrane attack complex.

In yet another aspect of the invention covers a method of preventing peritonitis mediated multi organ dysfunction in a subject, said method comprising step of: a) Identifying the subject; and b)Treating the subject with an effective dose of a composition consisting essentially of Withanone to prevent peritonitis mediated multi organ dysfunction.

The broader scope of applicability of the present invention will be apparent from the detailed description below. However, it should be understood that the detailed description and specific examples below, while indicating preferred embodiments of the invention, should not be construed as the limitations to the invention, and it is within the scope of those skilled in the art to make various changes and modifications, such as changing the concentration range of ingredients, derivatives/analogs of Withanone, are well within the spirit and scope of the invention from this detailed description.

### BRIEF DESCRIPTION OF DRAWINGS

FIG. 1 shows Intraperitoneal Cellular Influx, using Withanone at 0.5 mg/kg, 1 mg/kg, 2 mg/kg, and 4 mg/kg. *p<0.05, **p<0.01, ***p<0.001

FIGS. 2 and 3 shows effect of Withanone (2 mg/kg), Withania somnifera extract (100 mg/kg) on CD4+ and CD8+ T cells, compared to Zymosan and Naïve control . *p<0.01, **p<0.001

FIG. 4 shows effect of Withanone (2 mg/kg), Withania somnifera extract (100 mg/kg) compared to Zymosan and Naive control on the expression levels of cytokines. *p<0.01, **p<0.001

FIG. 5 shows effect of Withanone (2 mg/kg), Withania somnifera extract (100 mg/kg) compared to Zymosan and Naïve control on the expression of Myeloperoxidase assay. *p<0.01

FIG. 6 shows effect of Withanone (2 mg/kg), Withania somnifera extract (100 mg/kg) compared to Zymosan and Naïve control on the expression of Membrane attack complex (MAC). *p<0.01, **p<0.001

FIG. 7 shows effect of Withanone (2 mg/kg), Withania somnifera extract (100 mg/kg) compared to Zymosan and Naïve control on the expression of complement activation product, C3b. *p<0.01

FIG. 8 shows effect of Withanone (2 mg/kg), Withania somnifera extract (100 mg/kg) compared to Zymosan and Naïve control on the expression of Procalcitonin. *p<0.01, **p<0.001

### DESCRIPTION OF PREFERRED EMBODIMENTS

### Selected Definitions

All the terms used in this application carry ordinary meaning as known in the prior art unless otherwise specified. Few other specific definitions used in this invention are explained below, which applies throughout this specification. Claims provide broader definition unless and otherwise specified.

Withanone used in this invention is at least 99% pure quantified by HPLC, and isolated from the roots of Withania somnifera. (Anjali et al. Multifunctional neuroprotective effect of Withanone, a compound from Withania somnifera roots in alleviating cognitive dysfunction; Cytokine2017—Incorporated herein by reference). All the extracts used in the invention are isolated from the roots of Withania somnifera.

Therapeutically managing or management refers to a condition of effectively ameliorating conditions disclosed in the invention. Control experiments in the invention includes a Zymosan treated and naïve control.

The invention in general covers a method and composition for use in therapeutic management of peritonitis in a subject. The invention also covers a composition and method for preventing peritonitis mediated multi organ dysfunction in a subject. The invention further covers a method and composition consisting essentially of Withanone for therapeutically managing inflammation in a subject.

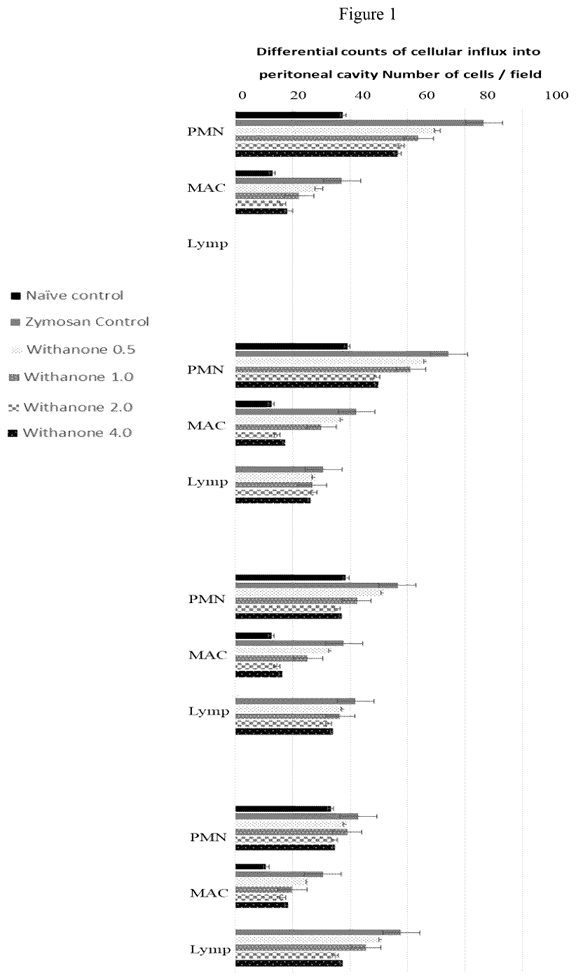

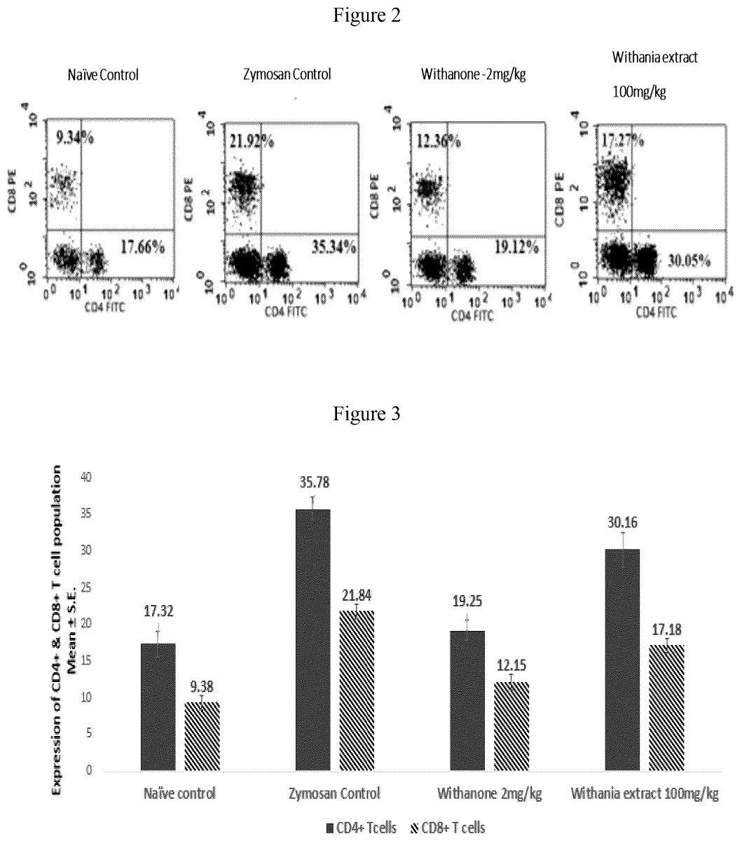

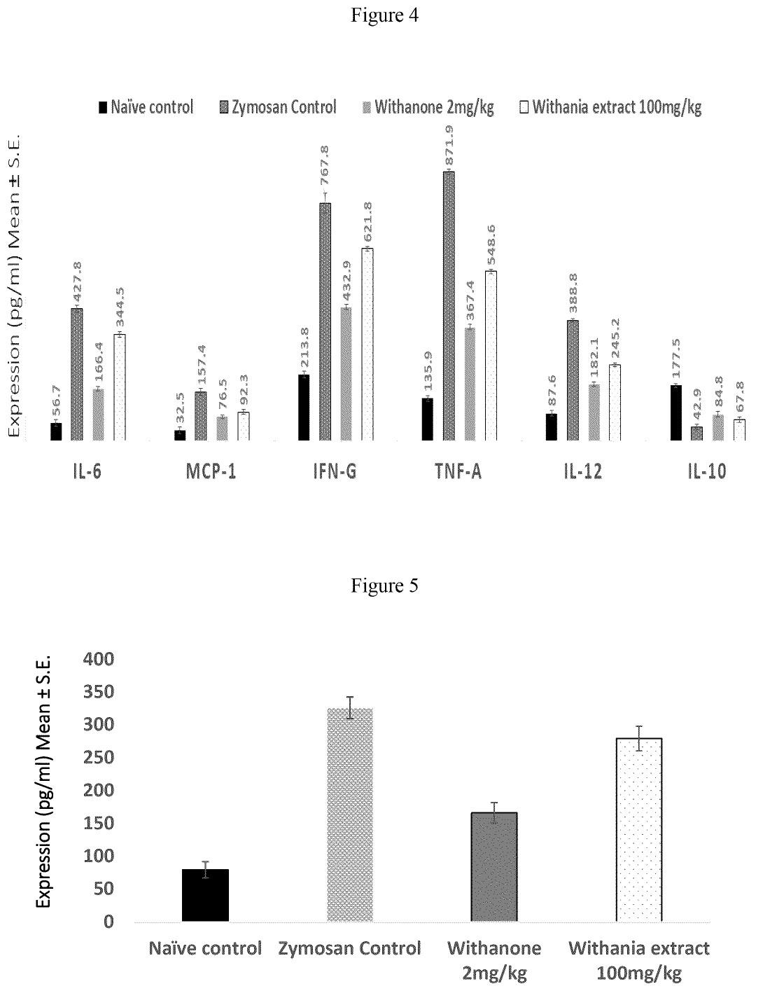

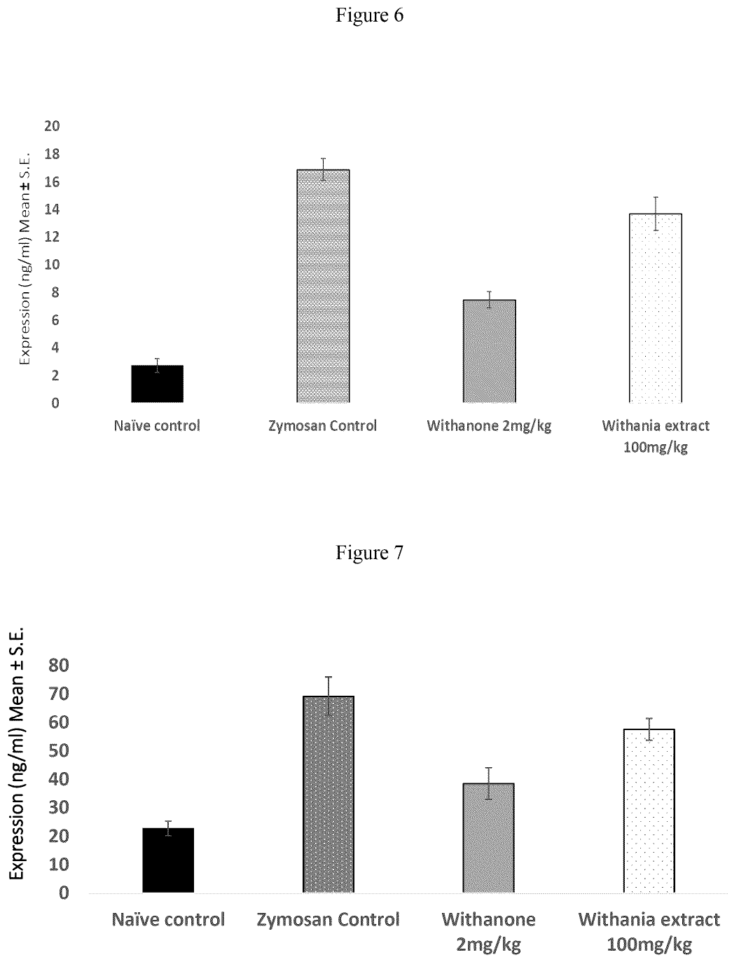

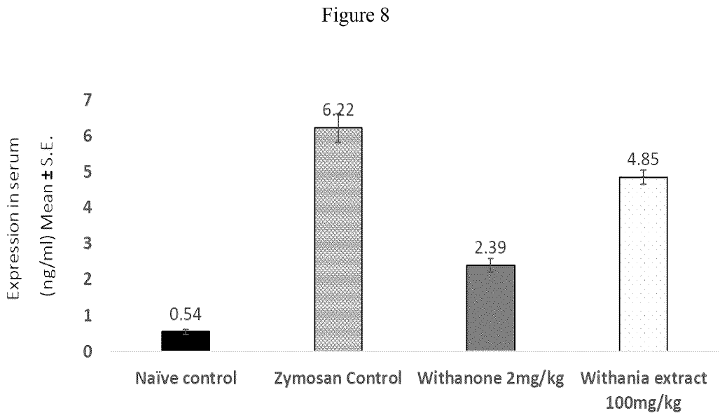

In the most preferred embodiment, the invention discloses a composition consisting essentially of Withanone for use in the therapeutic management of peritonitis in a mammal. In a related aspect of this embodiment, the management of peritonitis is brought about by inhibiting markers associated with inflammation, preventing bacterial translocation, inhibiting complement activation, decreasing circulating levels of procalcitonin and inhibiting the formation of membrane attack complex (MAC). Further aspect of this embodiment the markers associated with inflammation are selected from the group consisting of polymorphonuclear leukocytes, macrophages, lymphocytes, CD4+, CD8+ T cells, TNF-α, IL-1β, IL-6, IL-10, MCP-1, IFN-γ, myeloperoxidase, and IL-12p70. In a related aspect of this embodiment, cellular influx of polymorphonuclear leukocytes, macrophages, lymphocytes is inhibited by Withanone preferably at 0.5 mg/kg, or preferably by 1 mg/kg, or preferably by 2 mg/kg, or preferably by 4 mg/kg (Example 3, FIG. 1). In related aspect of this embodiment, Withanone inhibits CD4+ preferably by at least 40%, or preferably by 45%, or preferably by 50% compared to the Zymosan control. In another aspect of this embodiment, Withanone inhibits CD8+ at least 40%, or preferably 45%, or preferably 50% compared to the Zymosan control (Example 5, FIGS. 2, 3). In another related aspect of this embodiment, Withanone inhibits cytokines IL-6, MCP-1, IFN-γ, TNF-α, IL-12, IL-10 preferably by 40%, or preferably by 45%, or preferably by 50%, or preferably by 55%, or preferably by 60% compared to the Zymosan control (Examples 1, 2, 6, Table 1, 2, FIG. 4). in yet another aspect of this embodiment, withanone decreased the expression levels of myeloperoxidase (MPO) by at least 40%, or preferably 45%, or preferably 50% compared to the Zymosan control (Example 8, FIG. 5). In another related aspect of this embodiment, the inhibition of complement activation is brought about by decreasing the expression of C3b by Withanone in 40%, or preferably by 45% compared to Zymosan control (Example 9, FIG. 7), and yet another aspect of this embodiment, withanone decreased the expression of membrane attack complex (MAC) by 45%, or preferably by 50%, or preferably by 55%, or preferably by 60% compared to the Zymosan control (Example 7, FIG. 6). In another related aspect of the embodiment, expression level of procalcitonin is decreased by Withanone preferably by 45%, or preferably by 50%, or preferably 55%, or preferably by 60% compared to the Zymosan control (Example 9, FIG. 8). In yet another aspect of the embodiment, the mammal is human. In another aspect of this embodiment, the effective dose of Withanone is in the range of 1 mg/kg to 20 mg/kg. It is within the scope of the invention to explore other possible dosages.

In another most preferred embodiment of the invention, the invention discloses a method of therapeutic management of peritonitis in a mammal, said method comprising steps of a) identifying the mammal with peritonitis; and b) administering an effective dose of a composition consisting essentially of Withanone to said mammal, to alleviate the symptoms of peritonitis. In related aspect of this embodiment, management of peritonitis is brought about by inhibiting markers associated with inflammation and inhibiting the formation of membrane attack complex (MAC). Further aspect of this embodiment the markers associated with inflammation are selected from the group consisting of polymorphonuclear leukocytes, macrophages, lymphocytes, CD4+, CD8+ T cells, TNF-α, IL-6, IL-10, MCP-1, IFN-γ, myeloperoxidase, and IL-12p70. In a related aspect of this embodiment, cellular influx of polymorphonuclear leukocytes, macrophages, lymphocytes is inhibited by Withanone preferably at 0.5 mg/kg, or preferably by 1 mg/kg, or preferably by 2 mg/kg, or preferably by 4 mg/kg (Example 3, FIG. 1). In another related aspect of this embodiment, Withanone inhibits CD4+ by at least 40%, or preferably by 45%, or preferably by 50% compared to the Zymosan control. In another aspect of this embodiment, Withanone inhibits CD8+ by at least 40%, or preferably 45%, or preferably 50% compared to the Zymosan control (Example 5, FIGS. 2, 3). In another related aspect of this embodiment, Withanone inhibits cytokines IL-6, MCP-1, TNF-α, IL-12, IL-10 preferably by 40%, or preferably by 45%, or preferably by 50%, or preferably by 55%, or preferably by 60% compared to the Zymosan control (Examples 1,2, 6; Table 1, 2, FIG. 4). In yet another aspect of this embodiment, Withanone decreased the expression levels of myeloperoxidase (MP( )by at least 40%, or preferably 45%, or preferably 50% compared to the Zymosan control (Example 8, FIG. 5). In another aspect of this embodiment, the inhibition of complement activation is brought about by decreasing the expression of C3b by Withanone in 40%, or preferably by 45% compared to Zymosan control (Example 9, FIG. 7), and yet another aspect of this embodiment, withanone decreased the expression of membrane attack complex (MAC) by 45%, or preferably by 50%, or preferably by 55%, or preferably by 60% compared to the Zymosan control (Example 8, FIG. 6). In another aspect of the embodiment, expression level of procalcitonin is decreased by Withanone preferably by 45%, or preferably by 50%, or preferably 55%, or preferably by 60% compared to the Zymosan control (Example 9, FIG. 8). In yet another aspect of the embodiment, the mammal is human. In another aspect of this embodiment, the effective dose of Withanone is in the range of 1 mg/kg to 20 mg/kg. It is within the scope of the invention to explore other possible dosages.

In another most preferred embodiment of the invention, the invention discloses a composition consisting essentially of Withanone for therapeutically managing inflammation in a subject. Further aspect of this embodiment the markers associated with inflammation are selected from the group consisting of polymorphonuclear leukocytes, macrophages, lymphocytes, CD4+, CD8+ T cells, TNF-α, IL-6, IL-10, MCP-1, IFN-γ, myeloperoxidase, and IL-12p70. In a related aspect of this embodiment, cellular influx of polymorphonuclear leukocytes, macrophages, lymphocytes is inhibited by Withanone preferably at 0.5 mg/kg, or preferably by 1 mg/kg, or preferably by 2 mg/kg, or preferably by 4 mg/kg (Example 3, FIG. 1). In related aspect of this embodiment, Withanone inhibits CD4+ at least 40%, or preferably by 45%, or preferably by 50% compared to the Zymosan control. In another aspect of this embodiment, Withanone inhibits CD8+ at least 40%, or preferably 45%, or preferably 50% compared to the Zymosan control (Example 5, FIGS. 2, 3). In a related aspect of this embodiment, Withanone inhibits cytokines IL-6, MCP-1, IFN-γ, TNF-α, IL-10 preferably by 40%, or preferably by 45%, or preferably by 50%, or preferably by 55%, or preferably by 60% compared to the Zymosan control (Examples 1,2,6, Table 1, 2, FIG. 4). In yet another aspect of this embodiment, withanone decreased the expression levels of myeloperoxidase (MPO) by at least 40%, or preferably 45%, or preferably 50% compared to the Zymosan control (Example 8, FIG. 5). In another aspect of this embodiment, the inhibition of complement activation is brought about by decreasing the expression of C3b by Withanone in 40%, or preferably by 45% compared to Zymosan control (Example 9, FIG. 7), and yet another aspect of this embodiment, withanone decreased the expression of membrane attack complex (MAC) by 45%, or preferably by 50%, or preferably by 55%, or preferably by 60% compared to the Zymosan control (Example 8, FIG. 6). In another aspect of the embodiment, expression level of procalcitonin is decreased by Withanone preferably by 45%, or preferably by 50%, or preferably 55%, or preferably by 60% compared to the Zymosan control (Example 9, FIG. 8). In another aspect of this embodiment, the subject is a mammal or mammalian cells. In related aspect of this embodiment, the mammal is a human. In another aspect of this embodiment, the effective dose of Withanone is in the range of 1 mg/kg to 20 mg/kg when used in-vivo, and 2 μg/ml to 150 μg/ml when used in-vitro. It is within the scope of the invention to explore other possible dosages.

In yet another most preferred embodiment, the invention discloses a method for therapeutically managing inflammation in a subject, comprising steps of
a) Identifying the subject with inflammation; and
b) Treating the subject with an effective dose of a composition consisting essentially of Withanone, to alleviate the expression of markers associated with inflammation and inhibiting the formation of membrane attack complex.

Further aspect of this embodiment the markers associated with inflammation are selected from the group consisting of polymorphonuclear leukocytes, macrophages, lymphocytes, CD4+, CD8+ T cells, TNF-α, IL-1β, IL-6, IL-10, MCP-1, IFN-γ, myeloperoxidase, and IL-12p70. In a related aspect of this embodiment, cellular influx of polymorphonuclear leukocytes, macrophages, lymphocytes is inhibited by Withanone preferably at 0.5 mg/kg, or preferably by 1 mg/kg. or preferably by 2 mg/kg, or preferably by 4 mg/kg (Example 3, FIG. 1). In another related aspect of this embodiment, Withanone inhibits CD4+ at least 40%, or preferably by 45%, or preferably by 50% compared to the Zymosan control. In another aspect of this embodiment, Withanone inhibits CD8+ at least 40%, or preferably 45%, or preferably 50% compared to the Zymosan control (Example 5, FIGS. 2, 3). In related aspect of this embodiment, Withanone inhibits cytokines IL-6, MCP-1, IFN-γ, TNF-α, IL-12, IL-10 preferably by 40%, or preferably by 45%, or preferably by 50%, or preferably by 55%, or preferably by 60% compared to the Zymosan control (Examples 1, 2, 6, Table 1, 2, FIG. 4). In yet another aspect of this embodiment, withanone decreased the expression levels of myeloperoxidase (MPO) by at least 40%, or preferably 45%, or preferably 50% compared to the Zymosan control (Example 8, FIG. 5). In another aspect of this embodiment, the inhibition of complement activation is brought about by decreasing the expression of C3b by Withanone in 40%, or preferably by 45% compared to Zymosan control (Example 9, FIG. 7), and yet another aspect of this embodiment, withanone decreased the expression of membrane attack complex (MAC) by 45%, or preferably by 50%, or preferably by 55%, or preferably by 60% compared to the Zymosan control (Example 8, FIG. 6). In another aspect of the embodiment, expression level of procalcitonin is decreased by Withanone preferably by 45%, or preferably by 50%, or preferably 55%, or preferably by 60% compared to the Zymosan control (Example 9, FIG. 8). In another aspect of this embodiment, the subject is a mammal or mammalian cells. In related aspect of this embodiment, the mammal is a human. In another aspect of this embodiment, the effective dose of Withanone is in the range of 1 mg/kg to 20 mg/kg when used in-vivo, and 2 μg/ml to 150 μg/ml when used in-vitro. It is within the scope of the invention to explore other possible dosages.

In yet another most preferred embodiment of the invention, the invention discloses a composition consisting essentially of Withanone for use in preventing peritonitis mediated multi organ dysfunction in a subject. In a related aspect of this embodiment, multi organ dysfunction is prevented by inhibiting bacterial translocation, decreasing the expression of inflammatory markers, decreasing the expression of C5a, C3b, whereby this inhibits formation of membrane attack complex. In a related aspect of this embodiment, bacterial translocation is studied in mesenteric lymph nodes, spleen, and liver. Further aspect of this embodiment, wherein aerobic bacteria is detected in the organs in the presence of Withanone, and Withania somnifera, Withanone clearly inhibited the bacterial translocation in the study group (Example 7, Table 3), In related aspect of this embodiment, wherein the inflammatory markers are selected from the group consisting of TNF-α, IL-1β, IL-6, IL-10, MCP-1. IFN-γ and Withanone inhibits cytokines IL-6, MCP-1, IFN-γ, TNF-α, IL-12, IL-10 preferably by 40%, or preferably by 45%, or preferably by 50%, or preferably by 55%, or preferably by 60% compared to the Zymosan control (Example 1, 2, 6, FIG. 4). In another aspect of this embodiment, the inhibition of complement activation is brought about by decreasing the expression of C3b by Withanone in 40%, or preferably by 45% compared to Zymosan control (Example 9, FIG. 7), and yet another aspect of this embodiment, withanone decreased the expression of membrane attack complex (MAC) by 45%, or preferably by 50%, or preferably by 55%, or preferably by 60% compared to the Zymosan control (Example 8, FIG. 6). In another aspect of this embodiment, the subject is a mammal and the effective dose of Withanone is 1 mg/kg to 20 mg/kg. It is within the scope of the invention to explore other possible dosages.

In another most preferred embodiment, the invention discloses a method of preventing peritonitis mediated multi organ dysfunction in a subject, said method comprising step of:
a) Identifying the subject; and
b) Treating the subject with an effective dose of a composition consisting essentially of Withanone to prevent peritonitis mediated multi organ dysfunction.
   In a related aspect of this embodiment, multi organ dysfunction is prevented by inhibiting bacterial translocation, decreasing the expression of inflammatory markers, decreasing the expression of C5a, C3b, whereby this inhibits formation of membrane attack complex. In a related aspect of this embodiment, bacterial translocation is studied in mesenteric lymph nodes, spleen, and liver. Further aspect of this embodiment, wherein aerobic bacteria is detected in the organs in the presence of Withanone, and Withania somnifera, Withanone clearly inhibited the bacterial translocation in the study group (Example 7, Table 3). In a related aspect of this embodiment, wherein the inflammatory markers are selected from the group consisting of TNF-α, IL-1β, IL-6, IL-10, MCP-1, IFN-γ and Withanone inhibits cytokines IL-6, MCP-1, TFN-γ, TNF-α, IL-12, IL-10 preferably by 40%, or more preferably by 45%, or more preferably by 50%, or more preferably by 55%, or more preferably by 60% compared to the Zymosan control (Examples 1, 2, 6 FIG. 4), In another aspect of this embodiment, the inhibition of complement activation is brought about by decreasing the expression of C3b by Withanone in 40%, or preferably by 45% compared to Zymosan control (Example 9, FIG. 7), and yet another aspect of this embodiment, withanone decreased the expression of membrane attack complex (MAC) by 45%, or preferably by 50%, or preferably by 55%, or preferably by 60% compared to the Zymosan control (Example 8, FIG. 6). In another aspect of this embodiment, the subject is a mammal and the effective dose of Withanone is 1 mg/kg to 20 mg/kg. It is within the scope of the invention to explore other possible dosages.

In all the related embodiments of the invention, the symptoms of peritonitis are selected from the group consisting of poor appetite; nausea and vomiting; abdominal ache, tenderness or distention; chills; fever; fluid in the abdomen; decreased urination; and disrupted bowel movement.

In all the embodiment of the invention, wherein the composition further comprises of stabilizing agents, bioavailability enhancers and antioxidants, pharmaceutically or nutraceutically or cosmeceutically accepted excipients and enhancers and suitably formulated to be administered orally in the form of tablets, capsules, syrups, gummies, powders, suspensions, emulsions, chewables, candies or eatables (Example 10). It is well within the scope of a person skilled in the art to come up with a suitable formulation for administration

### EXAMPLES

### Example 1: Intracellular TNF-α and IL-1β Estimation in Murine Neutrophils

Flowcytometric studies were carried out to determine the anti-inflammatory effect of active constituents of Withania somnifera extract on TNF-α and IL-1β cytokine expression in LPS activated murine neutrophils.

TNF-α and Interleukin 1 beta (IL-1β) cytokines play a major role in the pathogenesis of septic shock induced by LPS (Lipopolysaccharide) endotoxin injection. LPS is an important triggering factor for in vivo systemic inflammatory response. LPS activates neutrophils via engagement of TLR4 (Toll-like receptor 4), resulting in the induction of a characteristic proinflammatory phenotype and prolongation of cell lifespan. In brief, neutrophils extraction was performed by taking murine blood and centrifuging it at 250 g for 20 min. Middle layer was taken and layered with histopaque and incubated for 10 min. The samples were centrifuged at 700 g, the upper layer was taken and Facs Lysing solution added for the lysis of any traces of RBC. The samples were washed with PBS (200 g) for 10 min. Neutrophils were stimulated with. LPS (1 μg/ml) and incubated with test samples for 3 h, in CO2 incubator. The cells were then labelled with conjugated anti-mouse TNF-α monoclonal antibody and anti-mouse IL-1 beta antibody. Incubation of the cells was carried out for 30 min in dark. After washing with PBS (Phosphate buffered saline) the samples were acquired directly on Flowcytometer (BD-LSR, Beckton—Dickinson Biosciences, CA, USA). A fluorescence trigger was set on the PE (FL1) parameter of the gated neutrophil populations (10,000 events). Fluorescence compensation, data analysis, and data presentation was performed using Cell Quest Pro software (Beckton—Dickinson Biosciences, CA, USA). Withanone comparatively showed better inhibition of TNF-α and IL-1β at 53% and 32% respectively compared to the LPS control (Table 1). Withanone was much more effective than the whole extract of Withania somnifera extract, which in contrast upregulated TNF-α and IL-1β. While, the Withania somnifera extract was not effective in mitigating inflammation in peritonitis, withanone is an excellent anti-inflammatory agent.

TABLE 1
Percentage expression of TNF-α and IL-β in
LPS activated murine neutrophils

| | | | Percentage | Percentage |
|---|---|---|---|---|
| | | | TNF-α | IL-1β |
| | | | inhibition | inhibition |
| | | Concentration | compared to | compared to |
| Entry | Sample | (μg/ml) | LPS control | LPS control |
| 1 | LPS control | — | — | — |
| 2 | Withanolide | 5 | 15.01↑ | 7.24↑ |
| 3 | Withanone | 5 | 53.07↓ | 32.17↓ |
| 4 | Withaferin A | 5 | 38.46↓ | 16.82↓ |
| 5 | Withastramnolide | 5 | 23.11↑ | 20.45↑ |
| 6 | 27- | 5 | 11.18↑ | 9.22↑ |
| | Hydroxywithanone | | | |
| 7 | Withanoside IV | 5 | 29.08↑ | 19.17↑ |
| 8 | Withania | 100 | 32.52↑ | 23.77↑ |
| | Somnifera Extract | | | |

### Example 2: Extracellular In Vivo TNF-α and IL-1β Estimation in Serum From the Treated Mice

BALM male mice aged 6-8 weeks were maintained at 22±2° C. under 12/12 h light dark cycle. Mice received oral treatment of test drugs at graded doses (w/v) for 6 days, followed by intravenous injection of 1 mg/kg (Brieva et al. Immunoferon, a glycoconjugate of natural origin, inhibits LPS-induced TNF-α production and inflammatory responses. International Immunopharmacology (2001) 1, 1979-1987). Six mice were employed in each group and experiments were performed in triplicates. Blood was collected from retro-orbital plexus of the experimental animals and serum was separated. TNF-α, IL-1β production was evaluated by a commercial ELISA kit in serum from treated mice, 90 mins after LPS injection. Withanone comparatively showed better inhibition of TNF-α and IL-1β at 37.05% and 24.5% respectively compared to the LPS control (Table 2). Interestingly. Withanone was much more effective than the whole extract of Withania somnifera extract, which in contrast upregulated TNF-α and IL-1β.

TABLE 2
Percentage expression of TNF-α and IL-β in serum from treated mice

| | | | Percentage | Percentage |
|---|---|---|---|---|
| | | | TNF-α | IL-1β |
| | | | inhibition | inhibition |
| | | Concentration | compared to | compared to |
| Entry | Sample | (mg/kg p.o) | LPS control | LPS control |
| 1 | LPS control | — | — | — |
| 2 | Withanolide | 1 | 5.09↑ | 5.33↑ |
| 3 | Withanone | 1 | 37.05↓ | 24.5↓ |
| 4 | Withaferin A | 1 | 18.55↓ | 13.02↓ |
| 5 | Withastramnolide | 1 | 16.01↑ | 10.09↑ |
| 6 | 27- | 1 | 9.21↑ | 8.05↑ |
| | Hydroxywithanone | | | |
| 7 | Withanoside IV | 1 | 21.86↑ | 10.89↑ |
| 8 | Withania | 100 | 41.07↑ | 32.67↑ |
| | Somnifera Extract | | | |

### Example 3: Induction of Peritonitis: Acute study

Peritoneal inflammation was induced according to Doherty et al. in BALB/c male mice aged 6-8 weeks. Zymosan A (Sigma Chemical) was prepared freshly (2 mg/ml) in sterile, 0.9% w/v saline, and 0.5 ml was injected i.p. Zymosan A is a cell wall component of fungus, caused proliferative peritonitis with severe accumulation of inflammatory cells. At the selected time points 4, 12, 24 and 48 hrs, the peritoneal cavity was lavaged with 1.5 ml saline, and after a 30-s gentle manual massage, 0.5 ml exudate was retrieved, centrifuged at 3000 g for 3 min. 0.5 ml exudate was collected and used freshly for cell counts. Withanone dosed at 2 mg/kg p.o showed maximum effect in inhibiting cellular influx at different time intervals (FIG. 1).

### Example 4: Chronic Study

Peritoneal inflammation was induced by Zymosan A (Sigma Chemical) (Masashi et al. Zymosan, but Not Lipopolysaccharide, Triggers Severe and Progressive Peritoneal Injury Accompanied by complement Activation in a Rat Peritonitis Model) was prepared freshly in sterile, 0.9% w/v saline, and 0.5 ml was injected i.p. from day 0 to Day 5. The lymphocyte population in the peripheral blood was determined with a flow cytometer. Cells were phenotyped by flow cytometry with the following primary antibodies: CD4 (FITC) and CD8 (PE). Cells were gated broadly on all live lymphocyte cells, with only debris and multiple cell clusters being excluded. Analysis of mean fluorescence intensity was done and percentage cell population was evaluated. The test drugs were administered for 30 days after the induction of peritonitis. On 36^{th} day the peritoneal cavity was lavaged with 1.5 ml saline, and after a 30-s gentle manual massage, 0.5 ml exudate was retrieved, centrifuged at 3000 g for 3 min, and frozen at 220° C. prior to cytokine and chemotactic-factor assessment.

### Example 5: Peripheral Blood CD4+ and CD8+ T Cell Population

Flow cytometry enables the characterization of cells and subcellular organelles on the basis of size and granularity and a number of different parameters defined by fluorescent probes. The experimental animals were bled on day 36^{th} from the retro-orbital plexus to carry out immuno-phenotyping, of different T cell surface receptors. Specific molecules present on the cell surface define the lymphocytes functional state and capabilities. Fluorochrome labelled monoclonal antibodies directed against co-receptors CD4 and CD8 were used to quantify the lymphocyte subsets in a multiparametric flow cytometric assay. CD4 cells, also known as CD4 lymphocytes or T-helper cells) stimulates other immune cells (macrophages, B lymphocytes(B cells)), and CD8 cell fights infection. Ratio of CD4/CD8 is a measure of healthy immune system. FITC-labelled anti-mouse CD4 and PE-labelled CD8 monoclonal antibodies were used to determine the percentage of CD4+ and CD8+ T cells in the control and treated group of animals. FITC-labelled CD4 and PE-labelled CD8 monoclonal antibodies were added directly to 100 μL of whole blood. Tubes were incubated in the dark for 30 min at room temperature. Subsequently, 1X FACS lysing solution was added at room temperature with gentle mixing followed by incubation for 10 min. The samples were centrifuged at 300-400×g, the supernatant aspirated and the sample given three washings with phosphate buffer saline (pH 7.4). The resulting stained cell pellet was resuspended in 500 μL of phosphate buffer saline and was run on a flow cytometer. Analysis was done directly on a flow cytometer using Cell Quest Pro software. Withanone effectively inhibits CD4+ by 46% and CD8+ by 44% compared to T cell population of Zymosan control (FIG. 2, FIG. 3).

### Example 6: Cytometric Bead Array Immunoassay Analysis

The Cytometric Bead Array (Mouse Inflammation kit) was used to study cytokines in peritoneal fluid. A mouse inflammation kit was used to simultaneously detect mouse interleukin 6 (IL-6), IL-10, MCP-1, IFN-γ (interferon γ), TNF-α (tumour necrosis a), and IL-12p70. Populations with distinct fluorescent intensities (FL-3) are pre coated with antibodies specific for each cytokine. Briefly, a mixture of 6 capture bead populations (50 μl) with distinct fluorescence intensities (detected in FL3) coated with antibodies specific for the above cytokines was mixed with each sample/standard (50 μl). The mixture was incubated for 3 h in the dark at the room temperature. This mixture was washed and centrifuged at 500 g for 5 min and the pellet resuspended in 300 ml of wash buffer. Following acquisition of data by two colour cytometric analysis, the sample results were analysed using CBA software (BD Biosciences). The Flow cytometer was calibrated with setup beads and 3000 events were acquired for each sample. Standard curves were generated for each cytokine using the mixed cytokine standard provided by the kit. Individual cytokine concentration ratios were indicated by their fluorescent intensities and was determined by interpolation from the corresponding standard curve. Withanone (2 mg/kg) effectively inhibited the cytokines (61% IL-6, 51% MCP-1, 44% IFN-γ, 58% TNF-α, 53% IL-12, 52% IL-10) compared to the whole extract of Withania somnifera (20% IL-6, 41% MCP-1, 19% IFN-γ, 37% TNF-α, 37% IL-12, 62% IL-10) (FIG. 4).

### Example 7: Bacterial Translocation

Intestine serves as a barrier in preventing gut bacteria and/or endotoxin from spreading to organs and tissues. It is also known that bacterial translocation occurs in critically ill or immunocompromised patients, and the physical disruption of mucosal barrier in vivo rodent models seem to be a key factor Tor translocation (Deitch et al. Effect of hemorrhagic shock on bacterial translocation, intestinal morphology, and intestinal permeability in conventional and antibiotic-decontaminated rats; Critical Care Medicine; 1990 18(5)). Bacterial translocation from the gut into the peritoneal cavity and organs was analyzed. The mesenteric lymph nodes, liver, and spleen of mice, study group of 30, were removed using sterile instruments. The organs were then homogenized in 1 ml of sterile cooked meat broth and 50 μl of organ homogenate was spread on blood agar plates to detect aerobic bacteria and Eosin Methylene blue agar plates to detect specially for E. coli. These plates were then incubated at 37° C. for 2 days. For facultative anaerobic bacteria 50 μl of above organ homogenate was spread on brain heart infusion agar plate and then incubated at 37° C. in 5% CO2 atmosphere for 2 days (Table 3). Withanone minimized bacterial translocation, wherein only 5 out of 30 mice showed bacterial translocation, compared to other study groups (Table 3).

TABLE 3
Study of bacterial translocation

| | Mesenteric Lymph Nodes | | | Spleen | | Liver | |
|---|---|---|---|---|---|---|---|
| Normal | E. Coli | 1/6 | 2/30 | 0/6 | 0/30 | 1/6 | 2/30 |
| Control | (Gram-negative) | | | | | | |
| | Lactobacillus | 1/6 | | 0/6 | | 1/6 | |
| | acidophilus | | | | | | |
| | (Gram positive) | | | | | | |
| | Enterococcus faecalis | 0/6 | | 0/6 | | 0/6 | |
| | (Gram positive) | | | | | | |
| | Klebsiella pneumoniae | 0/6 | | 0/6 | | 0/6 | |
| | (Gram-negative) | | | | | | |
| | Proteus mirabilis | 0/6 | | 0/6 | | 0/6 | |
| | (Gram-negative) | | | | | | |
| Zymosan | E.coli | 5/6 | 26/30 | 4/6 | 17/30 | 2/6 | 14/30 |
| Control | L. acidophilus | 6/6 | | 4/6 | | 3/6 | |
| | E. faecalis | 5/6 | | 3/6 | | 3/6 | |
| | k. pneumonia | 4/6 | | 3/6 | | 3/6 | |
| | p. mirabilis | 6/6 | | 3/6 | | 3/6 | |
| Withanone | E.coli | 2/6 | 5/30 | 1/6 | 3/30 | 1/6 | 3/30 |
| 2 mg/kg | L. acidophilus | 1/6 | | 1/6 | | 1/6 | |
| | E. faecalis | 2/6 | | 0/6 | | 1/6 | |
| | k. pneumonia | 0/6 | | 1/6 | | 0/6 | |
| | p. mirabilis | 0/6 | | 0/6 | | 0/6 | |
| Withania | E. coli | 3/6 | 14/30 | 2/6 | | 3/6 | 12/30 |
| Somnifera | L. acidophilus | 3/6 | | 3/6 | | 3/6 | |
| extract | E. faecalis | 2/6 | | 2/6 | 10/30 | 2/6 | |
| | k. pneumonia | 1/6 | | 2/6 | | 2/6 | |
| | P. mirabilis | 3/6 | | 1/6 | | 2/6 | |

### Example 8: Myeloperoxidase and Membrane Attack Complex

Circulating toxins can activate neutrophils or stimulate monocytes to release mediators contributing to the development of endotoxic shock. The stimulation of polymorphonuclear neutrophils results in a sudden increase in oxygen consumption, with the production of reactive oxygen species and the release of enzymes such as proteases (e.g., elastase) and myeloperoxidase (MPO). MPO is a pro-inflammatory enzyme stored in azurophilic granules of neutrophilic granulocytes Increased plasmatic MPO levels are a marker of neutrophil activation and degranulation and evaluating MPO levels or activity has been crucial in understanding its effect in inflammation (Pulli et al. measuring Myeloperoxidase Activity in Biological Samples; PLos One (2013) 8(7); e67976). On 36^{th} day the peritoneal cavity was lavaged with 1.5 ml saline, and after a 30-s gentle manual massage, 0.5 ml exudate was retrieved, centrifuged at 3000 g for 3 min. Myeloperoxidase was estimated using commercially available kits based on sandwich and competitive ELISA technique according to the manufacturers' instructions in the peritoneal exudate. The protein concentrations were carried out by means of colorimetric measurement at 450 nm on an ELISA plate reader by interpolation from a standard curve. Withanone dosed at 2 mg/kg decreased the expression levels of MPO by at least 50% compared to the Zymosan control. In contrast, Withania somnifera whole extract dosed at 100 mg/kg decreased the expression levels of MPO by 14% compared to the Zymosan control (FIG. 5).

Assembly of Membrane attack complex (MAC) results from the complex pathway starting from C5 convertase cleaving C5 to form C5a and C5b. C5a, a potent anaphylatoxin, promotes leucocyte activity and upregulation of immune response. It is believed that C5b initiates MAC assembly on membranes, proximal to activation. MAC serves as an important innate immune effector in the complement terminal pathway, which forms cytotoxic pores on the surface of microbes (Bayly-Jones et al. The mystery behind membrane insertion: a review of the complement membrane attack complex. Phil. Trans. R. Soc. B 372: 20160221). On 36^{th} day the peritoneal cavity was lavaged with 1.5 ml saline, and after a 30-s gentle manual massage, 0.5 ml exudate was retrieved, centrifuged at 3000 g for 3 min. MAC was estimated using commercially available kits based on sandwich and competitive ELISA technique according to the manufacturers' instructions in the peritoneal exudate. The protein concentrations were carried out by means of colorimetric measurement at 450 nm on an ELISA plate reader by interpolation from a standard curve. Withanone dosed at 2 mg/kg decreased the expression levels of MAC by 56% compared to the Zymosan control. In contrast, Withania somnifera whole extract dosed at 100 mg/kg decreased the expression levels of MPO by at least 7% compared to the Zymosan control (FIG. 6).

### Example 9: Complement Activation and Procalcitonin

Complement activation is a system of more than 30 proteins in the plasma and on cell surfaces. The activation takes place by three pathways, classical, lectin, and alternative. It is considered that generation of C3 convertase cleaves C3 into anaphylatoxin C3a and the opsonin C3b, which is the convergence point for the complement activation pathway. It is the thioester linkage of C3b which allows covalent linkage with the hydroxyl groups of proximal carbohydrates and proteins, this tags the microorganisms as foreign leading to further activation of complement activation, and thereby assembly of membrane attack complex (Dunkelberger et al. Complement and its role in innate and adaptive immune responses; Cell Research: 20 (2010) 34-5). Activation of the complement system protects against microorganisms in the peritoneal cavity. However, unregulated complement activation causes tissue damage in many diseases. Peritonitis and subsequent peritoneal sclerosis are associated with inflammation and complement activation. The peritoneal mesothelial cells become activated by Zymosan and secrete a battery of chemokines and cytokines that result in upregulation of the complement system, activation of the fibrin system and the recruitment of cells into the peritoneal cavity. The deposition of fibrin localizes infection so that the irritants become easy targets for neutrophils and macrophages. The blood was collected from retro-orbital plexus from the animals of all groups. The blood was allowed to stand for 2 hrs without addition of anti-coagulant. Serum was separated following centrifugation (8000 rpm) for 10 mins. Estimation of Complement activation was carried out using an enzyme linked immunosorbent assay. Withanone dosed at 2 mg/kg inhibited the complement activation product, C3b by 44% compared to the Zymosan control and performed better than Withania somnifera whole extract dosed at 100 mg/kg, which inhibited 9% compared to the Zymosan control (FIG. 7).

Procalcitonin is a precursor for calcitonin, a peptide hormone involved in calcium homeostasis. In response to pro-inflammatory stimulus of bacterial origin, the procalcitonin level rises. The blood was collected from retro-orbital plexus from the animals of all groups. The blood was allowed to stand for 2 hrs without addition of anti-coagulant. Serum was separated following centrifugation (8000 rpm) for 10 mins. Estimation of Procalcitonin expression was carried out using an enzyme linked immunosorbent assay. The expression level of procalcitonin in serum decreased in Withanone (2 mg/kg) treated sample by ˜60% compared to the Zymosan control, whereas the Withania somnifera whole extract (100 mg/kg) decreased the expression level by 22% compared to the zymosan treated control (FIG. 8).

Overall, withanone per se was more effective in managing peritonitis compared to Withania somnifera extract which includes different active molecules like Withanolide A, Withastramnolide, Withanone, 27-Hydroxy Withanone, Withaferin A, Withanoside IV. The doses for the animal study 1-20 mg/kg bodyweight, correspond to a human dose 5 mg-100 mg per day. Although Acharya et al. demonstrated use of Withania somnifera (WS) as a whole extract for treating endotoxin induced peritonitis, the activity may not be due the presence of withanone. The results clearly indicate that withanone decreases inflammation in peritonitis much better than the whole extract and individual actives of Withania somnifera including withaferin A. Thus, withanone, but not Withania somnifera extract, is effective for the management of peritonitis, making the finding novel and non-obvious.

Example 10: Formulations: The composition is formulated along with pharmaceutically/nutraceutically acceptable excipients, adjuvants, diluents, stabilizing agents, dispersible gums, bioavailability enhancers or carriers and administered orally in the form of tablets, capsules, syrups, gummies, powders, suspensions, emulsions, chewable, candies or eatable.

In a related aspect the bioavailability enhancer is selected from the group of piperine (BioPerine®), quercetin, garlic extract, ginger extract, and naringin. In another related aspect, the stabilizing agent is selected from the group consisting rosmarinic acid, butylated hydroxyanisole, butylated hydroxytoluene, sodium metabisulfite, propyl gallate, cysteine, ascorbic acid and tocopherols. In yet another related aspect, the dispersible gums are selected from the group consisting of Agar, Alginate, Carrageenan, Gum Arabic, Guar Gum, Locust Bean Gum, Konjac Gum, Xanthan Gum and Pectin.

Tables 4-8 provide illustrative examples of nutraceutical formulations containing bisdemethoxycurcumin

TABLE 4
Tablet
Active Ingredients
Withanone
Excipients
Microcrystalline cellulose, Colloidal silicon dioxide,
Magnesium stearate, BioPerine ®, Polyvinylpyrrolidone/starch/
Hydroxy propyl methyl cellulose, Hydroxy propyl cellulose

TABLE 5
Capsule
Active Ingredients
Withanone
Excipients
Microcrystalline cellulose, BioPerine ®

TABLE 6
Powder
Active Ingredients
Withanone
Excipients
BioPerine ® ,

TABLE 7
Gummy formulation
Active Ingredients
Withanone
Excipients
BioPerine ®, Gelatin (270 Bloom Mesh 10),
Refined Sugar, Glucose Corn Syrup,
Citric Acid, Lactic Acid, Water, Natural
Mango Flavor M38630, Tartaric Acid, Refined Sugar

TABLE 8
Candy formulation
Active Ingredients
Withanone
Excipients
BioPerine ®, Sucrose, Liquid Glucose,
Flavoring agent, Menthol, Acidulants
(Citric acid/Tartaric Acid/Maleic Acid), Purified water

The above formulations are merely illustrative examples, any formulation containing the above active ingredient intended for the said purpose will be considered equivalent.

Other modifications and variations of the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention and is to be interpreted only in conjunction with the appended claims.

We claim,:

1. A method of therapeutic management of peritonitis in a mammal, said method comprising steps of a) identifying the mammal with peritonitis; and b) administering an effective dose of a composition consisting essentially of Withanone to said mammal, to alleviate the symptoms of peritonitis.

2. The method as in claim 1, wherein management of peritonitis is brought about by inhibiting markers associated with inflammation, preventing bacterial translocation, inhibiting complement activation, decreasing circulating levels of procalcitonin and inhibiting complement activation and the formation of membrane attack complex.

3. The method as in claim 1, wherein the symptoms of peritonitis are selected from the group consisting of poor appetite; nausea and vomiting; abdominal ache, tenderness or distention; chills; fever; fluid in the abdomen; decreased urination; and disrupted bowel movement.

4. The method as in claim 2, wherein the markers associated with inflammation are selected from the group consisting of polymorphonuclear leukocytes, macrophages, lymphocytes, CD4+, CD8+ T cells, TNF-α, IL-1β, IL-6, IL-10, MCP-1, IFN-γ, myeloperoxidase, and IL-12p70.

5. The method as in claim 2, wherein inhibition of complement activation is brought about by decreasing the expression of C5a, C3b, whereby this inhibits formation of membrane attack complex.

6. The method as in claim 1, wherein the composition further comprises pharmaceutically or nutraceutically accepted excipients and enhancers and administered orally in the form of tablets, capsules, syrups, gummies, powders, suspensions, emulsions, chewables, candies or eatables.

7. The method as in claim 1, wherein the mammal is human.

8. A method for therapeutically managing inflammation in a subject, comprising steps of
a) Identifying the subject with inflammation; and
b) Treating the subject with an effective dose of a composition consisting essentially of Withanone, to alleviate the expression of markers associated with inflammation and inhibiting the formation of membrane attack complex.

9. The method as in claim 8, wherein the subject is a mammal or mammalian cells.

10. The method as in claim 8, wherein management of inflammation is brought about by inhibiting markers associated with inflammation, inhibiting complement activation, and inhibiting complement activation and the formation of membrane attack complex

11. The method as in claim 10, wherein the markers associated with inflammation are selected from the group consisting of polymorphonuclear leukocytes, macrophages, lymphocytes, CD4+, CD8+ T cells, TNF-α, IL-1β, IL-6, IL-10. MCP-1, IFN-γ, myeloperoxidase, and IL-12p70.

12. The method as in claim 10, wherein inhibition of complement activation is brought about by decreasing the expression of C5a, C3b, whereby this inhibits formation of membrane attack complex.

13. The method as in claim 8, wherein the mammal is human.

14. The method as in claim 8, wherein the composition further comprises pharmaceutically or nutraceutically accepted excipients and enhancers.

15. A method of preventing peritonitis mediated multi organ dysfunction in a subject, said method comprising step of:
a) Identifying the subject; and
b) Treating the subject with an effective dose of a composition consisting essentially of Withanone to prevent peritonitis mediated multi organ dysfunction.

16. The method as in claim 15, wherein multi organ dysfunction is prevented by inhibiting bacterial translocation, decreasing the expression of inflammatory markers, decreasing the expression of C5a, C3b, whereby this inhibits formation of membrane attack complex.

17. The method as in claim 15, wherein the inflammatory markers are selected from the group consisting of TNF-α, IL-1β, IL-6, IL-10, MCP-1, IFN-γ.

18. The method as in claim 15, wherein the composition further comprises pharmaceutically or nutraceutically accepted excipients and enhancers and administered orally in the form of tablets, capsules, syrups, gummies, powders, suspensions, emulsions, chewables, candies or eatables.

19. The method as in claim 15, wherein the subject is a mammal.
